(19) United States
(12) Patent Application Publication
(10) Pub. No.: US 2007/0073368 A1
(43) Pub. Date: Mar. 29, 2007

(54) Shape blankets

(75) Inventors: Karl Cazzini, Orchard Park, NY (US); Joel T. Jusiak, Holland, NY (US)

Correspondence Address: Kevin D. McCarthy;Roach Brown McCarthy & Gruber, P.C., 420 Main Street- 1620 Liberty Building, Buffalo, NY, 14202, US

(73) Assignee: Gaymar Industries, Inc.

(21) Appl. No.: 11/235,934

(22) Filed: Sep. 27, 2005

(51) Int. Cl. A61F 7/00

(52) U.S. Cl. 607/104

(57) ABSTRACT

The present invention is directed to a blanket. The blanket has a first sheet and a second sheet attached together at least at the periphery of each sheet. The first sheet comprises a non-metallic, polymeric containing shape conforming material. The shape conforming material conforms to the shape of a patient that it is applied to. The shape conforming material becomes shape conformant when it is exposed to a predetermined temperature or a predetermined condition.

### FIELD OF THE INVENTION

The present invention is directed to blankets. The blanket can be an overlay blanket, a convective blanket, a hypothermia blanket and/or a hyperthermia blanket.

### BACKGROUND OF THE INVENTION

The present invention is directed to incorporating known non-metallic, polymeric containing shape conforming materials into known blanket designs to obtain an efficient transfer of thermal energy to and/or maintenance of thermal energy from a patient.

Those shape conforming materials are known in some industries and have been incorporated into materials associated with
(a) Novel Gaskets or Seals: Moisture-activated sealing devices for the construction industry would be convenient to deploy on-site in areas such as roofing, guttering, caulking, and other film barrier applications;
(b) Medical and Healthcare Products: Delivery of pharmaceutical drugs, especially as capsule coatings for enteric delivery, offers new possibilities to the formulator. Shrinkable bandages, wound dressings, or veterinary applications also could use these film characteristics;
(c) Packaging Films: Improved packaging films can be managed, especially those applications requiring triggered shrink-wraps or the elimination after use of bulky items. Example: Industrial shrink-wraps, food packaging, horticultural applications, and film coatings for box or paperboard.
(d) Electronic or Mechanical Switching Systems: These films may be used in alarm systems for detection of moisture or aqueous leaks requiring immediate notification or corrective action. Example: High-humidity detection, leak detection, or high-water-level detection in tankage systems.

Gaymar is unaware of any non-metallic, polymeric containing shape conforming materials being incorporated into blankets or blanket designs. It is Gaymar's opinion that such shape conforming blankets are needed in the marketplace to ensure that the thermal energy is efficiently transferred, obtained, and/or maintained in association with the patient underlying and/or overlying the blanket.

### SUMMARY OF THE INVENTION

The present invention is directed to a blanket. The blanket has a first sheet and a second sheet attached together at least at the periphery of each sheet. The first sheet comprises a non-metallic, polymeric containing shape conforming material. The shape conforming material conforms to the shape of a patient that it is applied to. The shape conforming material becomes shape conformant when it is exposed to a predetermined temperature or a predetermined condition.

### BRIEF DESCRIPTION OF THE FIGURES

FIG. 1 illustrates a convective and/or fluid path blankets.

FIG. 2a illustrates a cross-sectional view of FIG. 1 taken along lines 2-2 if the blanket is a convective blanket embodiment.

FIG. 2b illustrates a cross-sectional view of FIG. 1 taken along lines 2-2 if the blanket is a fluid path blanket embodiment.

FIG. 3 illustrates a conventional blanket design.

FIGS. 4 to 7 illustrate cross-sectional views of various embodiments of the present invention.

### DETAILED DESCRIPTION OF THE INVENTION

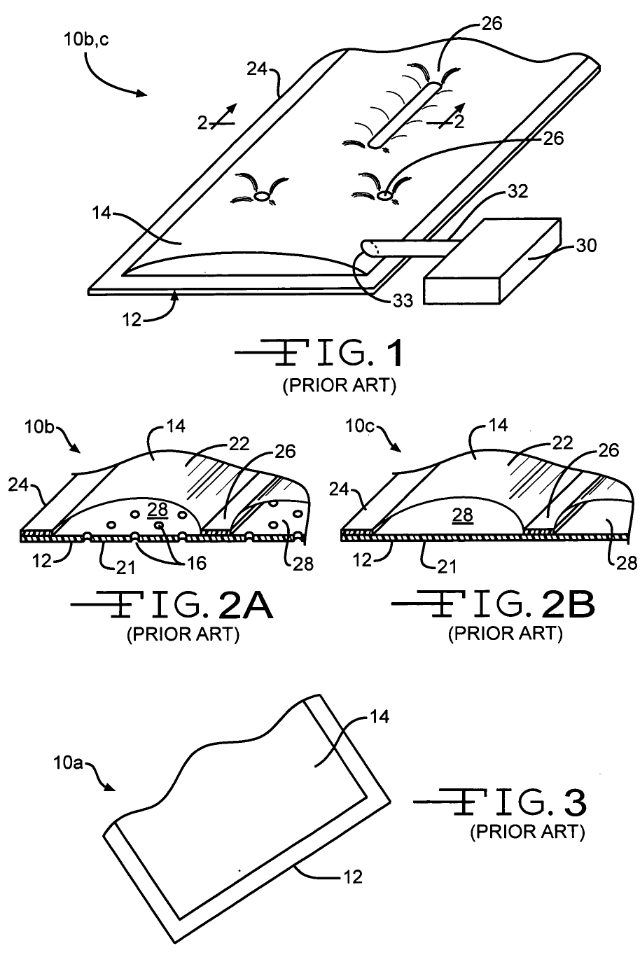

The present invention contains a blanket 10 (which is used generically to describe each blanket embodiment 10a, 10b, 10c), as illustrated in FIGS. 1, 2a, 2b, and 3. The blanket 10 has a contacting surface 12 and a non-contacting surface 14. The contacting surface 12 contacts a patient's clothing, skin and/or combinations thereof, while the non-contacting surface 14 does not normally contact the patient's clothing, skin and/or combinations thereof. In some embodiments the non-contacting surface 14 and the contacting surface 12 can be interchanged as illustrated in FIGS. 1 and 2b, and sometimes not as illustrated in FIG. 2a—due to the position of holes 16 on the contacting surface 12.

There are numerous types of blankets. There are conventional wool, fleece, cotton, flannel, down and electrical thermal blankets 10a as illustrated in FIG. 3. These blankets can be purchased where conventional bedding and/or conventional blankets are sold. These blankets 10a are referred to in this application as conventional blankets.

Other types of blankets include convective blankets 10b as illustrated in both FIGS. 1 and 2a. Since 1992, Gaymar Industries, Inc. manufactures and sells convective warming blankets to prevent or treat hyperthermia and/or hypothermia. Gaymar's convective blankets 10b, some of which are referred to as Thermacare quilts, feature an inflatable quilt-like structure. The convective blankets attach two sheets 21, 22, preferably, of the same amount of flexible, lightweight material around their periphery 24 and sometimes at various spots 26 along their surfaces to form a cavity 28. The cavity 28 forms a path for the desired medium to follow to reach a desired location in the blanket 10b, c. The path could have any design to accomplish that objective. Common path designs include and are not limited to serpentine, counter-flow serpentine paths, finger-like extensions and variations and combinations thereof.

In operation, heated (or cooled) air flows onto a patient's body from the cavity 28 through holes 16 on the undersurface 12, 21 of Gaymar's blankets, but the blankets do not form a self-supporting or Quonset hut-like structure around the patient. Instead, Gaymar's convective blankets lie flat when inflated on a flat surface and rest substantially on a patient when in use. In addition, Gaymar's convective blankets feature air distribution paths (the cavities) 28 designed for optimal effectiveness to controlling and/or manipulating a patient's temperature, with ample dimensions to accommodate all patients and perforated areas for better patient access.

The heated (or cooled) air provided to the convective blanket can be provided by any type of machine 30 that provides the desired forced medium—for convective blankets 10b that medium is air—at the desired temperature to the convective blanket. An example of such a device 30 and not to be limited to such is Gaymar's Thermacare patient warming (cooling) system unit. The Thermacare patient warming (cooling) system unit provides effective patient warming (cooling) adaptable to a variety of procedures. The system unit features a lightweight, portable, warm (cool)-air blower unit offering three temperature settings for the OR/ICU and four for the PACU. The unit's flexible, covered hose 32 is lightweight and easy to clean, and attaches easily and securely to the blanket's medium opening 33. The system unit 30 can be mounted on an IV pole, foot end of bed or on a portable stand. The blower unit is used with Thermacare quilts, which come in five versatile, easy-to-use sizes. The Thermacare system, with its fast and convenient setup, also meets NFPA standards for patient safety.

Other types of blankets that can be used in the present invention receive a fluid (like the convective blanket does) and the fluid circulates through and out of the blanket 10c through conduits 32. Examples of these blankets include and are not limited to Gaymar's DHP 600 Series Hyper/Hypothermia blanket, DHL520 Rapr Round Blanket and T-pads. These blankets 10c are similar to Gaymar's convective blankets except there are no holes in the undersurface. Instead, the fluid traverses through a path 28 defined by areas where the two sheets are joined together and once the entire fluid circulates through the path, the fluid is released through an outlet (not shown) to a different or in some case the same conduit 32. Hereinafter, such blankets 10c are referred to as fluid path blankets.

The fluid source for the fluid path blankets is any device 30 that can provide the desired fluid at the desired temperature to the fluid path blanket through conduits. The fluid can be and not limited to water or any other aqueous containing liquid that is not deleterious to the blanket 10c and the patient if there is ever a leak. An example of such a device and not to be limited to such a device is Gaymar's Medi-therm® III Hyper/Hypothermia System. The Medi-therm system delivers reliable and effective patient warming and cooling to fluid path blankets. The system combines time-proven technology with microprocessor accuracy and offers features that provide more effective controlled warming and/or cooling.

For this application, the term “blanket” by itself, refers to any of the above-identified blankets. If a particular type of blanket is identified, then that sentence and/or paragraph is limited to that particular blanket design.

For this application, Gaymar has no interest in altering the fundamental principals of how these various types of blankets function. Instead, Gaymar desires to improve the function of these blankets by substituting at least a portion of the blanket's material—topside, underside and/or both, but normally the underside (the portion of the blanket that contacts the majority of the patient's skin and/or clothing)—with a non-metallic, polymeric containing shape conforming material 50. When the blanket 10 conforms to the shape, or at least a part of the shape, of the user, the blanket 10 has increased surface area on the user. Increased surface contact area means the blanket is providing an efficient and desired thermal energy transfer and/or maintenance to the user. That is a desirable outcome of the present invention over the known prior art blanket embodiments.

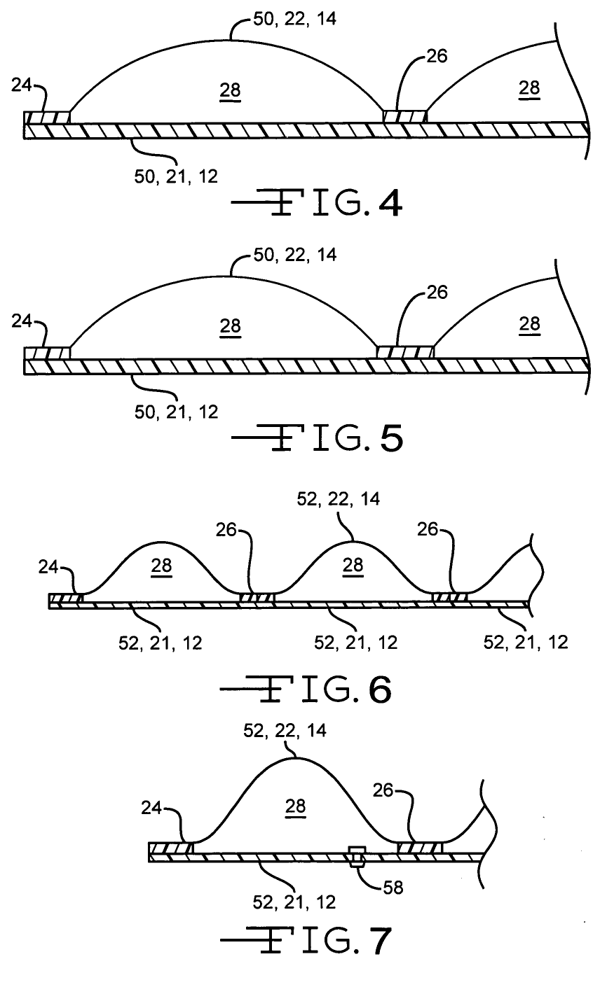

Even with the shape conforming material 50, the blankets are fabricated in substantially the manner as previously manufactured. That is because the shape conforming material contains at least some polymeric material, like and not limited to a styrene polymer system, that allows itself to be manufactured in the same way as other blankets are manufactured. More importantly, the shape conforming material 50 can comprise the entire blanket material as illustrated in FIG. 4, the contacting surface 12 of the blanket 10 as shown in FIG. 5 being attached to the original type of polymeric containing blanket material 52, a portion of the contacting surface of the blanket 10 as shown in FIG. 6.

The shape conforming materials 50 that can be incorporated into the blankets 10 are known to be used in different applications as cited above—not blankets. The shape conforming material 50 are polymers whose qualities have been altered to give them dynamic shape “memory” properties. Using thermal stimuli, shape conforming materials 50 can exhibit a radical change from a rigid polymer to a very elastic state, then back to a rigid state again. In its elastic state, the shape conforming material 50 will recover its “memory” shape if left unrestrained. However, while pliable the shape conforming material 50 can be stretched, folded or otherwise conformed to other shapes, tolerating up to 200% elongation.

While manipulated, the shape conforming material 50 can be cooled below a preset temperature value and therefore returned to a rigid state, maintaining its manipulated shape indefinitely. This manipulation process can be repeated many times without degradation, and many of these polymers can be tailored with shape memory properties.

One of many types of this shape conforming material 50 is sold under the trademark Veriflex™ by CRG Industries. By no means is the present invention limited to this particular brand or type. The Veriflex brand of shape conforming material 50 currently functions on thermal activation customizable from −20° F. to 520° F. Shape conforming material 50 changes between rigid and elastic states by way of thermal stimuli. The change takes place at what is referred to as the glass transition temperature (T_{g}).

Unlike shape memory alloys, shape conforming material 50 is a polymer that exhibits a radical change from a normal rigid polymer to very stretchy elastic and back on command, a change which can be repeated without degradation of the material. The “memory,” or recovery, quality comes from the stored mechanical energy attained during the reconfiguration and cooling of the material.

While in its elastic state, shape conforming material 50 will recover its cast, or cured, shape if left unrestrained. Otherwise, the shape conforming material 50 may be reconfigured or manipulated into other shapes, tolerating up to 200% elongation. Shape conforming material 50 heated above its transition temperature can be stretched, folded, rolled, twisted, or bent. At any point, the shape conforming material 50 can be cooled to maintain its altered shape indefinitely, until once again it is brought above its transition temperature. Above its transition temperature, the shape conforming material 50 recovers its “memorized” shape very quickly.

Shape conforming material 50 is not simply an elastomer, nor simply a plastic. It exhibits characteristics of both materials, depending on its temperature. While rigid, the shape conforming material 50 demonstrates the strength-to-weight ratio of a rigid polymer. While pliable, the shape conforming material 50 has the flexibility of a high-quality, dynamic elastomer.

Alternatively, the shape conforming material 50 can be a water-shrinkable film. An example of such a film and not limited to such is manufactured by Kimberly-Clark. These films shrink without heat, change shape and/or size, and conform as desired to objects—in this embodiment to a patient.

These films provide two distinct operational modes. Structural integrity as manufactured is maintained until a triggering mechanism initiates a rapid shrinkage to a new pre-determined shape or form a patient's body part for this invention. The new form determines a second set of characteristics.

The film's characteristics can be tailored for specific end uses by selection and quantity of the water dispersible and the elastomeric components. Triggering mechanisms may be customized, for instance, by pH change and/or moisture may be built into film design to give controlled release of active materials under specific conditions.

The film shrinkage results from the unique two-component system, where an elastomer is held in tension by a water-dispersible polymer during processing. This tension is locked-in and maintained until contact with moisture or aqueous fluids, whereupon tension is released. Processing options can allow shrinkage to be either asymmetric or bi-directional as desired.

The films are prepared by mixing the water dispersible and the elastomeric polymers, then forming a film by traditional extrusion or casting. These well-known process operations result in efficient production, high capacity, low cost operation and require minimal expertise.

Principles for producing at least one embodiment of the water-shrinkable films have been disclosed in a series of patents issued to Kimberly-Clark Corporation: U.S. Pat. No. 5,932,068, issued Aug. 3, 1999 to Farrington, Jr. et al.; U.S. Pat. No. 5,672,248 issued to Wendt et al.; U.S. Pat. No. 5,048,589, issued Sep. 17, 1991 to Cook et al.; U.S. Pat. No. 6,436,234, issued Aug. 20, 2002 to Chen et al.; U.S. Pat. No. 5,888,347, issued Mar. 30, 1999 to Engel et al.; U.S. Pat. No. 5,429,686, issued Jul. 4, 1995 to Chiu et al.; U.S. Pat. No. 6,120,642, issued Sep. 19, 2000 to Lindsay et al.; U.S. Pat. No. 6,398,910, issued Jun. 4, 2002 to Burazin et al.; U.S. Pat. No. 5,779,860, issued Jul. 14, 1998 to Hollenberg et al.; U.S. Pat. No. 6,436,234, issued Aug. 20, 2002 to Chen et al.; and PCT publications WO 03/40464 and WO 03/40470, both published May 15, 2003 by Burazin et al.; WO 03/59139 by Chen et al., published Jul. 24, 2003.

Alternatively the fluid within the blanket contacts a conductive material 58 as illustrated in FIG. 7 that (1) penetrates into the cavity 28 where the fluid circulates within the blanket, and (2) is exposed to the exterior surface of the blanket that is designed to contact the patient so the conductive material transfers the thermal energy of the fluid through the conductive material to the patient. The conductive material 58 is equivalent to a rivet-like metallic instrument that prevents (and/or decreases the chances of) leaking from the cavity 28.

Using the shape conforming material will decrease the chance the blanket will have to be taped (or adhered) on to the patient to ensure the proper positioning of the blanket. Adhesives and/or tape can occlude the flow of the fluid in the convective and fluid path blankets. Such occlusion can be deleterious to providing the desired thermal energy from the blanket to the user.

Another problem with using tape is that tape does not allow the user to easily reposition the blanket without subjecting the blanket to rips and tears.

Another problem with tape is that some patients have skin reactions to the tape and the adhesive of the tape.

Using a shape conforming material solves these problems.

Although a particular preferred embodiment of the invention has been illustrated and described in detail for illustrative purposes, it will be recognized that variations or modifications of the disclosed apparatus, including the rearrangement of parts, lie within the scope of the invention defined by the claims.

The embodiments of the invention in which an exclusive property or privilege is claimed are defined as follows:

We claim:

1. A blanket comprising:
a first sheet and a second sheet attached together at the periphery of each sheet;
the first sheet comprises a shape conforming material that conforms to the shape of a patient that the shape conforming material is applied to when the shape conforming material is exposed to a predetermined temperature or a predetermined condition.

2. The blanket of claim 1 wherein the first sheet and the second sheet form a cavity.

3. The blanket of claim 2 wherein the cavity receives a fluid from a fluid source that provides the fluid to the cavity through a conduit.

4. The blanket of claim 3 wherein the fluid is air.

5. The blanket of claim 4 wherein the first sheet contains apertures that allows the air to be directed to the patient.

6. The blanket of claim 3 wherein the fluid is a liquid.

7. The blanket of claim 6 wherein the liquid is water.

8. The blanket of claim 6 wherein the first sheet and the second sheet do not contain apertures.

9. The blanket of claim 3 wherein the fluid is warmed to a desired temperature above the temperature of the patient.

10. The blanket of claim 3 wherein the fluid is cooled to a desired temperature below the temperature of the patient.

11. The blanket of claim 3 wherein the fluid is maintained at a normothermic temperature in relation to the patient.

12. The blanket of claim 1 wherein the predetermined condition is a fluid contacting the shape conforming material.

13. The blanket of claim 1 wherein the shape conforming material contains a polymeric material.

14. The blanket of claim 1 wherein the blanket is selected from the group consisting of a fluid flow blanket, a convective blanket, a conventional blanket, and combinations thereof.

15. The blanket of claim 1 wherein the blanket is a fluid flow blanket and/or convective blanket and the fluid within the blanket contacts a conductive material that (1) penetrates into a cavity where the fluid circulates within the blanket, and (2) is exposed to the exterior surface of the blanket that is designed to contact the patient so the conductive material transfers the thermal energy of the fluid through the conductive material to the patient.

16. The blanket of claim 1 wherein the second sheet comprises the shape conforming material.

17. The blanket of claim 1 wherein the first sheet and the second sheet are the same material.

18. The blanket of claim 1 wherein the first sheet and the second sheet are different materials.

19. The blanket of claim 1 wherein the first sheet is comprised of two different materials.

20. A process of using a blanket comprising:
placing a first portion of a blanket over or under at least a part of a patient and a second portion of the blanket extends beyond at least a portion of one side of the user,
the blanket (a) is selected from the group consisting of a fluid flow design, a convective design or a combination thereof and (b) has a first sheet and a second sheet attached together at the periphery of each sheet;
the first sheet comprises a shape conforming material that conforms to the shape of a patient that the shape conforming material is applied to when the shape conforming material is exposed to a predetermined temperature or a predetermined condition and the first sheet is at least a part of the first portion;
exposing the shape conforming material to a predetermined temperature or a predetermined condition so the material is altered to conform to at least a portion of the patient.

21. The method of claim 20 wherein the exposing the shape conforming material to a predetermined temperature or a predetermined condition is accomplished by filling a cavity defined by the first sheet and the second sheet with a fluid.
